SEQUENCE LISTING (27 sequences)

SEQ ID NO: 1 - DNA, 20 bp, Artificial
  Forward, reverse and sequencing primers for each exon EGFR-Ex18
gctgaggtgacccttgtctc

SEQ ID NO: 2 - DNA, 20 bp, Artificial
  Forward, reverse and sequencing primers for each exon EGFR-Ex18
acagcttgcaaggactctgg

SEQ ID NO: 3 - DNA, 20 bp, Artificial
  Forward, reverse and sequencing primers for each exon EGFR-Ex18
tggagcctcttacacccagt

SEQ ID NO: 4 - DNA, 19 bp, Artificial
  Forward, reverse and sequencing primers for each exon EGFR-Ex19
cccagtgtccctcaccttc

SEQ ID NO: 5 - DNA, 20 bp, Artificial
  Forward, reverse and sequencing primers for each exon EGFR-Ex19
ccacacagcaaagcagaaac

SEQ ID NO: 6 - DNA, 20 bp, Artificial
  Forward, reverse and sequencing primers for each exon EGFR-Ex19
gctggtaacatccacccaga

SEQ ID NO: 7 - DNA, 20 bp, Artificial
  Forward, reverse and sequencing primers for each exon EGFR-Ex21
tgatctgtccctcacagcag

SEQ ID NO: 8 - DNA, 20 bp, Artificial
  Forward, reverse and sequencing primers for each exon EGFR-Ex21
tcaggaaaatgctggctgac

SEQ ID NO: 9 - DNA, 21 bp, Artificial
  Forward, reverse and sequencing primers for each exon EGFR-Ex21
ttcagggcatgaactacttgg

SEQ ID NO: 10 - DNA, 23 bp, Artificial
  Forward, reverse and sequencing primers for each exonPI3K CA-Ex9
gggaaaaatatgacaaagaaagc

SEQ ID NO: 11 - DNA, 22 bp, Artificial
  Forward, reverse and sequencing primers for each exonPI3K CA-Ex9
ctgagatcagccaaattcagtt

SEQ ID NO: 12 - DNA, 27 bp, Artificial
  Forward, reverse and sequencing primers for each exonPI3K CA-Ex9
tagctagagacaatgaattaagggaaa

SEQ ID NO: 13 - DNA, 20 bp, Artificial
  Forward, reverse and sequencing primers for each exonPI3K CA-Ex20
ctcaatgatgcttggctctg

SEQ ID NO: 14 - DNA, 21 bp, Artificial
  Forward, reverse and sequencing primers for each exonPI3K CA-Ex20
tggaatccagagtgagctttc

SEQ ID NO: 15 - DNA, 22 bp, Artificial
  Forward, reverse and sequencing primers for each exonPI3K CA-Ex20
ttgatgacattgcatacattcg

SEQ ID NO: 16 - DNA, 27 bp, Artificial
  Forward, reverse and sequencing primers for each exon Ras ex2
ggtggagtatttgatagtgtattaacc

SEQ ID NO: 17 - DNA, 21 bp, Artificial
  Forward, reverse and sequencing primers for each exon Ras ex2
agaatggtcctgcaccagtaa

SEQ ID NO: 18 - DNA, 27 bp, Artificial
  Forward, reverse and sequencing primers for each exon Ras ex2
tcattatttttattataaggcctgctg

SEQ ID NO: 19 - DNA, 20 bp, Artificial
  Primers (designed to span a 100 to 200-bp non-repetitive region) for the EGFR gene, forward
gaattcggatgcagagcttc

SEQ ID NO: 20 - DNA, 19 bp, Artificial
  Primers (designed to span a 100 to 200-bp non-repetitive region) for the EGFR gene, forward
gacatgctgcggtgttttc

SEQ ID NO: 21 - DNA, 20 bp, Artificial
  Primer sequence for the Line-1 repetitive element, forward
aaagccgctcaactacatgg

SEQ ID NO: 22 - DNA, 21 bp, Artificial
  Primer sequence for the Line-1 repetitive element, reverse
tgctttgaatgcgtcccagag

SEQ ID NO: 23 - protein, 25 aa, Artificial
  Wild type EGFR
KTPQHVKITDFGLAKLLGAEEKEYH

SEQ ID NO: 24 - protein, 25 aa, Artificial
  G857R (Patient 13)
KTPQHVKITDFRLAKLLGAEEKEYH

SEQ ID NO: 25 - protein, 25 aa, Artificial
  L858R (NSCLC)
KTPQHVKITDFGRAKLLGAEEKEYH

SEQ ID NO: 26 - protein, 25 aa, Artificial
  Wild type BRAF
HEDLTVKIGDFGLATVKSRWSGSHQ

SEQ ID NO: 27 - protein, 25 aa, Artificial
  G595R (CRC)
HEDLTVKIGDFRLATVKSRWSGSHQ

(19) United States
(12) Patent Application Publication
(10) Pub. No.: US 2009/0269344 A1
(43) Pub. Date: Oct. 29, 2009

(54) Anti-EGFR antibody therapy based on an increased copy number of the EGFR gene in tumor tissues

(76) Inventors: Salvatore Siena, Mailand (IT); Mauro Moroni, Mailand (IT); Giovanna Marrapese, Mailand (IT); Andrea Sartore-Bianchi, Milan (IT); Silvio Veronese, Milan (IT); Marcello Gambacorta, Milan (IT); Silvia Benvenuti, Dusino San Michele (Asti) (IT); Federica Di Nicolantonio, La Loggia (Torino) (IT); Alberto Bardelli, Torino (IT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/911,380

(22) PCT Filed: Apr. 12, 2006
(86) PCT No.: PCT/EP06/03358

§ 371 Date: Apr. 1, 2008

(30) Foreign Application Priority Data
Apr. 14, 2005 (EP) 05008156.1

(51) Int. Cl. A61K 39/395; C12Q 1/68

(52) U.S. Cl. 424/138.1; 435/6

(57) ABSTRACT

The invention relates to an indidualized and personalized diagnosis and therapy of cancer based on specific molecular alterations which occur in specific tumor tissue of specific tumor patient populations. The therapy and diagnostic is based on the findings that proliferation and tumor growth of specific EGFR bearing tumor tissue expressing an amplified EGFR gene copy number may be abolished by anti-EGFR antibodies, while other individual molecular alterations such as mutations occurring in tumor tissues are unaffected by the same anti-EGFR antibody treatment.

### TECHNICAL FILED OF THE INVENTION

The invention relates to the diagnosis and therapy of tumors expressing higher levels of epidermal growth factor receptor (EGFR) by means of anti-EGFR antibodies. The invention relates furthermore to an individualized and personalized diagnosis and therapy of EGFR expressing cancer, based on specific molecular alterations which occur in specific tumor tissue of specific tumor patient populations. The therapy and diagnostic is based on the findings that proliferation and tumor growth of specific EGFR bearing tumor tissue displaying an amplified EGFR gene copy number may be abolished by anti-EGFR antibodies, while other individual molecular alterations occurring in tumor tissues, such as specific gene mutations, are unaffected by the same anti-EGFR antibody treatment.

### TECHNICAL BACKGROUND OF THE INVENTION

Biological molecules, such as monoclonal antibodies (MAbs) or other proteins/polypeptides, as well as small chemical compounds directed against various receptors and other antigens on the surface of tumor cells are known to be suitable for tumor therapy for more than twenty years. With respect to the antibody approach, most of these MAbs are chimerized or humanized to improve tolerability with the human immune system. MAbs or above-mentioned chemical entities specifically bind to their target structures on tumor cells and in most cases also on normal tissues and can cause different effects that dependent on their epitope specificity and/or functional characteristics of the particular antigen.

ErbB receptors are typical receptor tyrosine kinases that were implicated in cancer in the 1980s. Tyrosine kinases are a class of enzymes that catalyze the transfer of the terminal phosphate of adenosine triphosphate to tyrosine residues in protein substrates. Tyrosine kinases are believed, by way of substrate phosphorylation, to play critical roles in signal transduction for a number of cell functions. Though the exact mechanisms of signal transduction is still unclear, tyrosine kinases have been shown to be important contributing factors in cell proliferation, carcinogenesis and cell differentiation. Receptor type tyrosine kinases have an extracellular, a transmembrane, and an intracellular portion, while non-receptor type tyrosine kinases are wholly intracellular. Receptor-linked tyrosine kinases are transmembrane proteins that contain an extracellular ligand binding domain, a transmembrane sequence, and a cytoplasmic tyrosine kinase domain. The receptor-type tyrosine kinases are comprised of a large number of transmembrane receptors with diverse biological activity.

Different subfamilies of receptor-type tyrosine kinases have been identified. Implicated tyrosine kinases include fibroblast growth factor (FGF) receptors, epidermal growth factor (EGF) receptors of the ErbB major class family, and platelet-derived growth factor (PDGF) receptors. Also implicated are nerve growth Factor (NGF) receptors, brain-derived neurotrophic Factor (BDNF) receptors, and neurotrophin-3 (NT-3) receptors, and neurotrophin-4 (NT-4) receptors.

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-a), by the same tumor cells resulting in receptor activation by an autocrine stimulatory pathway (Baselga and Mendelsohn, Pharmac. Ther. 64:127-154 (1994)). The EGF receptor is a transmembrane glycoprotein which has a molecular weight of 170.000, and is found on many epithelial cell types. It is activated by at least three ligands, EGF, TGF-α (transforming growth factor alpha) and amphiregulin. Both epidermal growth factor (EGF) and transforming growth factor-alpha (TGF-a) have been demonstrated to bind to EGF receptor and to lead to cellular proliferation and tumor growth.

It has been demonstrated that anti-EGF receptor antibodies while blocking EGF and TGF-a binding to the receptor appear to inhibit tumor cell proliferation. In view of these findings, a number of murine and rat monoclonal antibodies against EGF receptor have been developed and tested for their ability inhibit the growth of tumor cells in vitro and in vivo (Modjtahedi and Dean, 1994, J. Oncology 4, 277). Humanized monoclonal antibody 425 (hMAb 425, matuzumab; U.S. Pat. No. 5,558,864; EP 0531 472) and chimeric monoclonal antibody 225 (cMAb 225), both directed to the EGF receptor, have shown their efficacy in clinical trials. The C225 antibody (cetuximab) was demonstrated to inhibit EGF-mediated tumor cell growth in vitro and to inhibit human tumor formation in vivo in nude mice. The antibody as well as in general all anti-EGFR antibodies, appear to act, above all, in synergy with certain chemotherapeutic agents (i.e., doxorubicin, adriamycin, taxol, and cisplatin) to eradicate human tumors in vivo in xenograft mouse models (see, for example, EP 0667165). Ye et al. (1999, Oncogene 18, 731) have reported that human ovarian cancer cells can be treated successfully with a combination of both chimeric mAb 225 and humanized mAb 4D5 which is directed to the HER2 receptor. Also a combination of matuzumab and cetuximab elicit a synergistic ant-tumor response (WO 04/32960). Another fully human anti-EGFR antibody is panitumumab (mAb ABX) (e.g. WO 98/50433, U.S. Pat. No. 6,235,883) developed by XenoMouse® technology.

Anti-epidermal growth factor receptor (EGFR) monoclonal antibodies, such as the chimeric monoclonal antibody c225 (cetuximab) and the fully human antibody panitumumab have shown remarkable clinical activity in about 10% of patients with chemotherapy-resistant metastatic colorectal cancer (mCRC). The molecular mechanisms underlying clinical responsiveness or resistance to these agents are presently unknown.

The therapeutic armamentarium against metastatic colorectal cancer (mCRC), the third most frequent cause of cancer deaths, has been recently enforced with monoclonal antibodies (mAbs) directed against the extra-cellular domain of the epidermal growth factor receptor (EGFR) (Erlichman and Sargent; 2004, N Engl J Med 351: 391-392). Among the anti-EGFR mAbs, the chimeric antibody cetuximab (Erbitux®) and the fully human antibody panitumumab have each demonstrated remarkable clinical activity in about 10% of patients with chemotherapy-resistant mCRC, but the molecular mechanisms underlying clinical responsiveness or resistance are presently unknown. Neither the diagnostic characteristics nor the degree of tumor EGFR expression evaluated by immunohistochemistry, correlate with clinical response (Saltz et al., 2004, J Clin Oncol 22: 1201-1208; Cunningham et al., 2004, N Engl J Med 351: 337-345; Hecht et al., 2004, Journal of Clinical Oncology, ASCO Annual Meeting Proceedings, Post-Meeting Edition). Understanding the molecular basis of clinical sensitivity or resistance to anti-EGFR moAbs may allow the identification of patients who are likely to benefit from cetuximab or panitumumab treatment. The biology of the EGFR has been studied in detail using both genetic and biochemical approaches (Ciardiello et al., 2003, Eur J Cancer 39: 1348-1354; Holbro et al., 2004, Annu Rev Pharmacol Toxicol 44: 195-217). The initial step of binding of a ligand to the extracellular portion of the receptor, promotes receptor dimerization and activation of its enzymatic activity, thus resulting in phosphorylation of the intracellular domain. Subsequently, cellular effectors bind to the phosphorylated residues of the intracellular domain and become activated, mainly through their relocalization to the plasma membrane. The small G protein Ras, the protein kinase Raf, and the lipid kinase PI3K play central roles as the intracellular mediators of the EGFR signaling. Genetic alterations of the EGFR and its effectors have been previously found in a variety of cancers (Bardelli et al., 2003, Science 300: 949; Vogelstein et al., 2004, Nat Med 10: 789-799; Bardelli et al., 2005, Curr Opin Genet Dev 15: 5-12).

Therefore, the hypothesis might come up that the clinical response to certain specific anti-EGFR antibodies such as cetuximab, panitumumab or matuzumab could be associated to molecular alterations affecting the EGFR or its immediate intracellular signal transducers.

In many cancers, such as mCRC neither the diagnostic characteristics of the tumor nor the degree of EGFR expression evaluated by immunohistochemistry, correlate with clinical response to EGFR antagonists, especially anti-EGFR antibodies, such as cetuximab, matuzumab (hMab 425) or panitumumab. Currently, therefore, most treated patients are exposed to the risk of ineffective therapy with undesired side effects. The efficacy of treatment of mCRC patients with anti-EGFR mAbs such as cetuximab, matuzumab or panitumumab represents a significant medical progress. However, treatment with anti-EGFR mAbs resulted in objective responses only in a fraction of patients in clinical studies involving chemorefractory patients, and there are no diagnostic tools to identify those who are likely to benefit from this therapy. As a result, most of treated patients are exposed to the risk of ineffective therapy with undesired side effects. Non-personalized therapies also result in enormous financial burden for health systems.

Therefore, there is a need to explain the differential response in patients to anti-EGFR monoclonal antibodies and to develop a strategy in order to identify cancer patients such as CRC patients likely to benefit from anti-EGFR antibody therapy. The molecular mechanisms underlying responsiveness or refractoriness of EGFR-expressing cancer cells to anti-EGFR mAbs are unknown. Therefore, there is a further need to provide diagnostic tools that show whether the response to anti-EGFR mAbs in cancer is correlated with biological predictors or markers including (i) mutations affecting the EGFR gene catalytic domain, (ii) mutations affecting the EGFR downstream signaling effectors; or (iii) amplification of the EGFR gene locus.

### SUMMARY OF THE INVENTION

It was found now according to this invention that the EGFR gene copy number displayed by tumor cells in tumor patients including chemorefractory patients is increased in about 89% of patients that elicit an objective response to said tumor and in only about 5.0% of patients with stable or progressive disease. Thereby, the mutational status of the EGFR catalytic domain and of its immediate downstream effectors PI3K, RAS, RAF does not correlate with said response.

According to the invention the same concentration of specific anti-EGFR antibodies, such as cetuximab, matuzumab or panitumumab that completely impaired proliferation of cells displaying an amplified EGFR gene copy number in cellular models of specific cancers, such as colorectal cancer, does not affect cells displaying no amplified EGFR copy number.

According to the invention, in patients suffering from specific cancers, preferably mCRC, the response to the treatment with specific anti-EGFR antibodies, like panitumumab, cetuximab or matuzumab (or any immunologically effective fragment or fusion protein thereof) can be significantly associated with the presence of an amplified copy number of the EGFR gene. In other words: those patients that are responsive or sensitive to anti-EGFR treatment have an increased copy number of the EGFR gene as compared with those patients that do not respond to the treatment with the same antibody in the same dose. Furthermore, it can be observed that an increased EGFR gene copy number is correlated with tumor shrinkage in patients and with a prolonged survival by treatment with said mAbs. In these patients, the tumor growth is likely to be driven predominantly by the EGFR pathway.

The amplified EGFR gene copy number can be measured according to the present invention by determining the ratio of the EGFR genes per nucleus and/or the ratio defined by the number of EGFR gene copies and CEP7 (chromosome 7 centromere probe). It has been found that, according to the invention, in tumor probes, wherein the ratio: EGFR gene copies/nucleus is >4, preferably in the range between 5.7 and 7.1, and/or the EGFR gene copies/CEP7 >2, the administration of an anti-EGFR antibody to a patient, from whom the tumor probe derives, is more effective than in patients having copy number ratios as defined lower than indicated. Patients having tumor cells displaying non-amplified or only slightly amplified EGFR gene copy numbers (ratios: 1 or <2) do not or not sufficiently respond to anti-EGFR antibody therapy.

This observation represents the first paradigm for a personalized targeted therapy of specific cancers, such as colorectal cancer, based on a specific molecular alteration. In order to administer said drugs in a patient most effectively, a tool is now provided to identify those patients most likely to benefit.

It was further found that there are a novel somatic mutation in the EGFR catalytic domain and a number of mutations in its immediate downstream effectors (such as KRAS and PI3KCA), these alterations do not correlate with responsiveness to anti-EGFR mAbs. These findings have a number of clinical and biological implications. In EGFR expressing and overexpressing cancer the response to anti-EGFR mAbs is probably less associated with mutations of the EGFR gene but rather with its increased/amplified copy number. These results suggest that treatments based on anti-EGFR antibodies are likely to work most efficiently against targets that are amplified rather then affected by point mutations. However, genetic alterations such as point mutations may contribute to the effectiveness and efficacy of anti-EGFR antibody treatment.

With respect to CRC, in particular; the proliferation of CRC cells with amplified EGFR gene copy number is abolished by anti-EGFR antibodies, such as cetuximab, while CRC cells with not amplified EGFR copy number are unaffected by same doses of the anti-EGFR monoclonal antibody. This indicates that cancer cells, especially CRC cells, with amplified EGFR gene are dependent and even addicted to this molecular alteration for their proliferation.

The present data also indicate that FISH (fluorescent in situ hybridization) measurement of the EGFR gene copy number could represent an experimental tool to identify patients with mCRC and other cancers who are likely to respond to anti-EGFR targeted mAbs. Moreover, contrary to semi-quantitative assays such as qPCR and Western blotting, in the case of overexpression of EGFR protein and increased EGFR gene copy number localized into discrete foci within the same tumor (FIG. 3), FISH analysis is not influenced by the concomitant presence of disomic tumor cells or normal stromal contaminants. Thus, a possible non homogeneous pattern of EGFR expression should be taken in account to explain lack of correlation between IHC and clinical response to mAbs (FIG. 3).

In other words: according to the present invention, it was further shown for the first time that those cancer patients, preferably mCRC patients, showing a clinical response to the administration of anti-EGFR mAbs such as cetuximab, matuzumab or panitumumab, which is significantly based on an increased EGFR gene copy number, may be selected and evaluated by using FISH analysis of individual tumor samples of said patients. In other word: patients that are positive for FISH have a higher gene copy number than patients who are negative for FISH. Thus, it can be concluded that patients displaying an increased EGFR copy number as analyzed by FISH have a better survival prediction than those patients showing a low gene copy number.

To sum up in a more general way the invention relates to the following subject-matters.
A method for treating tumors expressing EGF receptor (EGFR) in a patient by administering to said patient an anti-EGFR antibody in an amount which is sufficient to abolish the proliferation of said tumor cells having an amplified EGFR gene copy number.
A corresponding method, wherein said treatment is more effective compared to a treatment with same antibody in the same dose applied to tumor cells which do not elicit an amplified EGFR gene copy number.
A corresponding method, wherein said tumor cells additionally elicit molecular alterations or genetic mutations.
A corresponding method, wherein the amplified EGFR gene copy number is specific for said tumor.
A corresponding method, wherein the amplified EGFR gene copy number is specific for the individual cancer tissue profile of the patient.
A corresponding method, wherein said individual cancer tissue profile underlies molecular alterations.
A corresponding method, wherein said EGFR expressing tumor is colorectal cancer (CRC).
A corresponding method, wherein said colorectal cancer is metastatic (mCRC).
A corresponding method, wherein said anti-EGFR antibody is selected from the group of Mab 225 and Mab 425 in their murine, chimeric and humanized versions.
A use of an anti-EGFR antibody for the manufacture of a medicament for the treatment of cancer, which is based on EGFR expressing tumor cells having an amplified EGFR gene copy number, wherein said treatment is more effective compared to a treatment with same antibody in the same dose applied to tumor cells which do not elicit an amplified EGFR gene copy number.
A corresponding use of an anti-EGFR antibody, wherein said tumor cells additionally elicit molecular alterations or genetic mutations
A corresponding use, wherein said amplified EGFR gene copy number is specific for said tumor.
A corresponding use, wherein the amplified EGFR gene copy number is specific for the individual cancer tissue profile of the patient.
A corresponding use, wherein said individual cancer tissue profile underlies molecular alteration.
A corresponding use, wherein said EGFR expressing tumor is colorectal cancer (CRC).
A corresponding use, wherein said colorectal cancer is metastatic (mCRC).
A corresponding use, wherein said anti-EGFR antibody is selected from the group of Mab 225 and Mab 425 in their murine, chimeric and humanized versions.
A method for detecting and measuring in vitro the EGFR gene copy number of tumor tissue by using fluorescent in situ hybridization (FISH).
A use of fluorescent in situ hybridization (FISH) for in vitro identification of patients having tumors which respond to anti-EGFR antibodies.
A use of fluorescent in situ hybridization (FISH) for in vitro identification of patients having tumors which elicit an increased EGFR gene copy number
A corresponding use, wherein said tumor is colorectal cancer (CRC), preferably metastatic CRC.
A corresponding use, wherein said antibody is 225 or 424 in their murine, chimeric or humanized versions.
An in vitro method for detecting and analyzing whether a patient suffering from a cancer which overexpresses EGF receptor (EGFR), responds positively to the administration of an anti-EGFR antibody or an immunologically effective fragment thereof, the method comprising determining in vitro the EGFR gene copy number in a probe of tumor cells obtained from said patient and selecting said patient for administration with said anti-EFFR antibody if the tumor cells of said patient display an amplified copy number of EGFR genes.
A corresponding method, wherein the EGFR gene copy number is measured as ratio of the number of EGFR genes per nucleus.
A corresponding method, wherein said ratio is in the range between 4.0 and 8.2.
A corresponding method, wherein said ratio is in the range between 5.7 and 7.1.
A corresponding method, wherein the EGFR gene copy number is measured as ratio of the number of EGFR genes per CEP7.
A corresponding method, wherein said ratio is >2.
A corresponding method, wherein the EGFR gene copy number is measured by FISH analysis (fluorescence in situ hybridization).
A corresponding method, wherein said amplified EGFR gene copy number is specific for said tumor.
A corresponding corresponding method, wherein the amplified EGFR gene copy number is specific for the individual cancer tissue profile of the patient.
A corresponding method, wherein said individual cancer tissue profile underlies furthermore molecular alteration.
A corresponding method, wherein said molecular alteration is a point mutation within the EGFR gene.
A corresponding method, wherein said anti-EGFR antibody is selected from the group consisting of cetuximab (mAb c225), matuzumab (mAb h425) and panitumumab (mAb ABX) or their particular murine, chimeric or humanized versions.
A corresponding method, wherein the cancer is colorectal cancer (CRC), lung cancer, head and neck cancer and breast cancer.
The use of an anti-EGFR antibody, or an immunologically effective fragment thereof, for the manufacture of a medicament for the treatment of cancer in a patient, wherein said cancer overexpresses EGFR and displays an amplified EGFR gene copy number.
A corresponding use, wherein said EGFR gene copy number is measured as ratio of the number of EGFR genes per nucleus, and the value of this ratio is in the range between 4.0 and 8.2.
A corresponding use, wherein the value of said ratio is in the range between 5.7 and 7.1.
A corresponding use, wherein the treatment of said cancer is more effective compared to the treatment of a cancer patient with the same antibody in the same dose, wherein the cancer cells do not display an amplified EGFR copy number.
A corresponding use, wherein said amplified EGFR gene copy number is specific for said tumor.
A corresponding use, wherein the amplified EGFR gene copy number is specific for the individual cancer tissue profile of the patient.
A corresponding use, wherein said individual cancer tissue profile underlies genetic mutations.
A corresponding use, wherein said EGFR expressing tumor is colorectal cancer (CRC), lung cancer, breast cancer or head and neck cancer.
A corresponding use, wherein said anti-EGFR antibody is selected from the group consisting of cetuximab (mAb c225), matuzumab (mAb h425) and panitumumab (mAb ABX), or their particular murine, chimeric or humanized versions.
A method for detecting and measuring in vitro the EGFR gene copy number of tumor tissue, which overexpresses EGFR, by using fluorescent in situ hybridization (FISH) in an assay for determining the response of a cancer patient to the administration with an anti-EGFR antibody.

### SHORT DESCRIPTIONS OF THE FIGURES

FIG. 1—Missense heterozygous mutation in exon 21 (G857R) found in tumor of patient 13 (see also Table 2). The mutation affects a critical residue located in the activation loop of the EGFR kinase domain. The G857R is one amino acid apart from the recently described L858R mutation found in gefitinib and erlotinib responders in non-small cell lung cancer (NSCLC) (Lynch et al., 2004, N Engl J Med 350: 2129-2139.; Paez et al., 2004, Science 304: 1497-1500; Pao et al., 2004, Proc Natl Acad Sci USA 101: 13306-13311). A mutation affecting the analogous residue in the BRAF gene (G595R) was previously detected in colorectal cancer (CRC) (Wiley, Diaz, 2004, Jama 291: 2019-2020).

FIG. 2—Dual color fluorescent in situ hybridization assays for probes of EGFR gene (red) and chromosome 7 (CEP7; green). (A) Balanced disomy in normal colorectal mucosa; (B) Balanced disomy in tumor of patient 27; (C) Balanced polysomy in tumor of patient 3; (D) Amplification in tumor of patient 5.

FIG. 3—EGFR amplification and protein expression in tumor of patient 10. (A) conventional histology by hematoxylin and eosin staining. (B and C) EGFR gene amplification and protein overexpression by immunohistochemistry (Moroni et al., 2001, Clin Cancer Res 7:2770-5) in corresponding areas of same tumor.

FIG. 4—Molecular EGFR gene alterations and clinical response observed in patient 1. (A) Dual color fluorescent in situ hybridization assays for EGFR gene (red) and chromosome 7 (CEP7; green) probes showing increased copy number; (B) Relative amount of EGFR gene copies measured by quantitative PCR in tumour of patient 1, A431 cancer cell line (EGFR gene/nucleus 8.00; EGFR gene/CEP7 2.57) and non-malignant RPE (EGFR gene/nucleus 1.60; EGFR gene/CEP7 0.86) epithelial cell controls; (C) (D) Measurements of liver metastasis by CT before (highest diameter, L line 4.4 cm) and after (highest diameter, M line 2.3 cm) treatment with moAb in patient 1.

FIG. 5—Inhibition of colorectal cancer cell line proliferation by cetuximab. (A) Proliferation of colorectal cancer cell lines in three separate experiments (mean ±SD) in the presence of increasing concentrations of cetuximab. (B) Levels of EGFR protein measured by Western blot in individual cell lines. (C) EGFR gene copy number evaluated by FISH in colorectal cancer cell lines. (D) Dual color fluorescent in situ hybridization assays for the EGFR gene (red) and chromosome 7 (CEP7; green) probes showing increased copy number in the DiFi cell line.

### DETAILED DESCRIPTION

The term “copy numbed” is usually defined as the number of genes per genome. According to the invention the term “EGFR gene copy number” means the ratio of number of EGFR genes per nucleus. According to the invention this number varies from 1.0 to 8.2 or more preferably from 1.5 to 7.9

According to the invention the term “increased or amplified EGFR gene copy numbed” means that, in a relative perspective, above-defined ratio in cells of a specific tumor correlated to a specific patient (who responds to the anti-EGFR antibody treatment) is higher or amplified compared to the particular ratio in cells of a specific tumor correlated to another specific patient. In a more absolute perspective, the term means that the ratio (number EGFR gene/nucleus) is between 4.0 and 8.2, or 4.8 and 8.2, or 4.8 and 7.9, or 4.8 and 7.1, or 4.8 and 6.8, or 4.8 and 5.7. Preferably said ratio is between 5.7 and 8.2 and more preferably 5.7 and 6.8, and most preferably between 5.7 and 7.1.

According to these afore-mentioned values applicable to an “increased or amplified” EGFR gene copy number, the ratio values for a relatively decreased or lower or non-amplified copy number presented by tumor cells of patients, which do not or not effectively or positively respond to the treatment with anti-EGFR antibodies are in the range between 1.65 and 2.0, or 1.7 and 1.9.

The EGFR gene copy number or the ratio: EGFR gene copies/nucleus is associated with the ratio EGFR gene copies/chromosome 7 centromere probe (CEP7). According to the invention this EGFR gene/CEP7 ratio is in patients clearly responding to anti-EGFR antibody treatment >2, whereas the ratio in patients who do not respond is usually approximately 1.

“Missense heterozygous mutation” means according to the invention a mutation that changes a codon for one amino acid into a codon specifying another amino acid occurring in one of the two alleles of a gene.

The term “In-frame deletion” means according to the invention a mutation that changes the reading frame of an mRNA by deleting nucleotides

“FISH (fluorescence in situ hybridization)” means according to the invention a hybridization of cloned DNA to intact chromosomes, where the cloned DNA has been labeled with a fluorescent dye. This is a general method to assign chromosomal location, gene copy number (both increased and decreased), or chromosomal rearrangements

Tumors from patients (31) with mCRC who achieved objective response, stable disease or progressive disease after treatment with cetuximab or panitumumab are screened for genetic alterations in the EGFR gene or its immediate intracellular effectors. Specifically, the EGFR gene copy number and the mutational profile of the EGFR catalytic domain can be determined as well as the exons in the KRAS, BRAF, and PI3KCA genes where mutations occur more frequently in mCRC.

Mutational Analysis of the EGFR Tyrosine Kinase Domain

To identify the molecular basis underlying response to matuzumab, panitumumab or cetuximab in mCRC, the mutational status of the region is evaluated corresponding to the catalytic domain of the EGFR gene in tumor specimens of patients with various clinical outcomes after treatment with these mAbs.

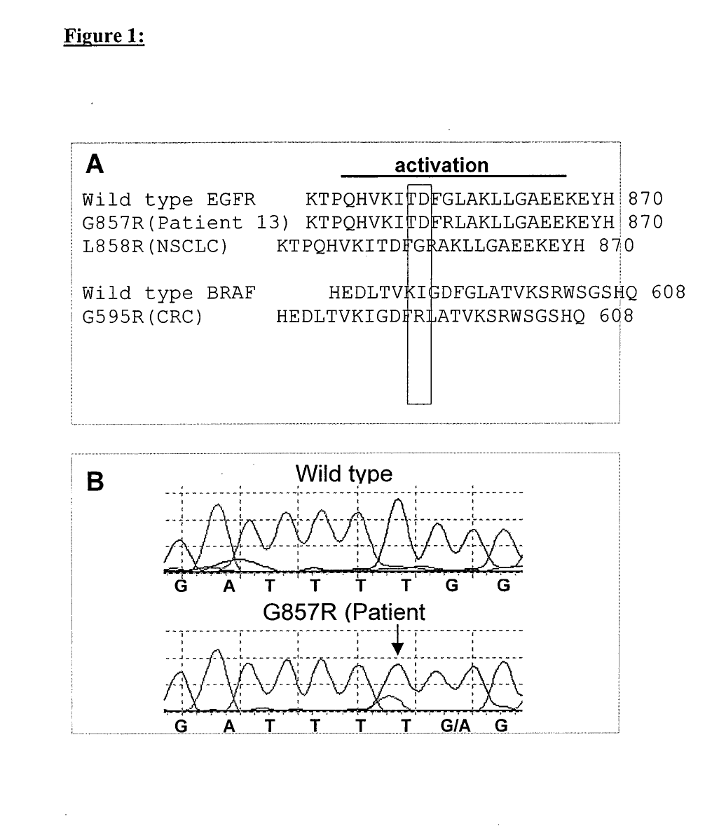

Sequencing of EGFR exons 18, 19 and 21 does not reveal somatic mutations with the exception of one patient with stable disease for 24 weeks (Tables 1 and 2). This patient displays a missense heterozygous mutation in exon 21 (G857R) affecting a residue located in the activation loop, a region that is critical for catalysis (FIG. 1). The G857R mutation is one amino acid apart from the recently described L858R activating mutation found in gefitinib and erlotinib responders in lung cancer (Lynch et al, 2004; N Engl J Med 350; 2129-2139; Paez et al., 2004, Science 304: 1497-1500; Pao et al., 2004, Proc Natl Acad Sci USA 101: 13306-13311)

Interestingly, a mutation affecting the analogous residue in the BRAF gene (G595R) has been previously detected in colorectal cancers (FIG. 1) (Rajagopalan et al., 2002, Nature 418: 934).

Based on present findings, it appears clear that the main molecular mechanism underlying response to mAb therapy are not mutations in the EGFR catalytic domain. Therefore, it is considered that alterations in the EGFR gene copy number might be responsible for the observed antibody response.

Mutational Analysis of EGFR Intracellular Effectors

At least three intracellular molecules (KRAS, BRAF, and PI3KCA) involved in EGFR signaling can be activated by point mutations in colorectal cancers. According to this invention it was assessed whether the mutational status of the corresponding genes is correlated with the clinical response to anti-EGFR antibodies, such as cetuximab, matuzumab or panitumumab. For each of the three genes, the exons are analyzed where mutations occur with the highest frequencies in colorectal cancers (KRAS exon 2, BRAF exon 15, PI3KCA exons 9 and 20). The nucleotide sequence corresponding to each exon can be amplified from tumor-extracted genomic DNA and directly sequenced. Although activating mutations can be identified in the KRAS gene (G12V, G12D, G12S, and G13D), PI3KCA gene (E545K, H1047R) and BRAF (E599V), they do not correlate with clinical response to anti EGFR mAbs (RAS exon-2: p=0.675; PI3K exon-9: p=0.3; PI3K exon-20: p=1; BRAF exon-15: p=1; all these mutations: p=0.44) (Tables 1 and 2).

Copy Number Analysis of the EGFR Gene by FISH Analysis

It can be shown that in mCRC there is no correlation between the levels of EGFR protein expression measured by immunohistochemistry (IHC) and clinical response to anti-EGFR mAbs. These results, together with the lack of correlation with the mutational status of the EGFR and its downstream effectors, may lead to the hypothesis that response to panitumumab, cetuximab or matuzumab may be associated with amplification of the EGFR gene.

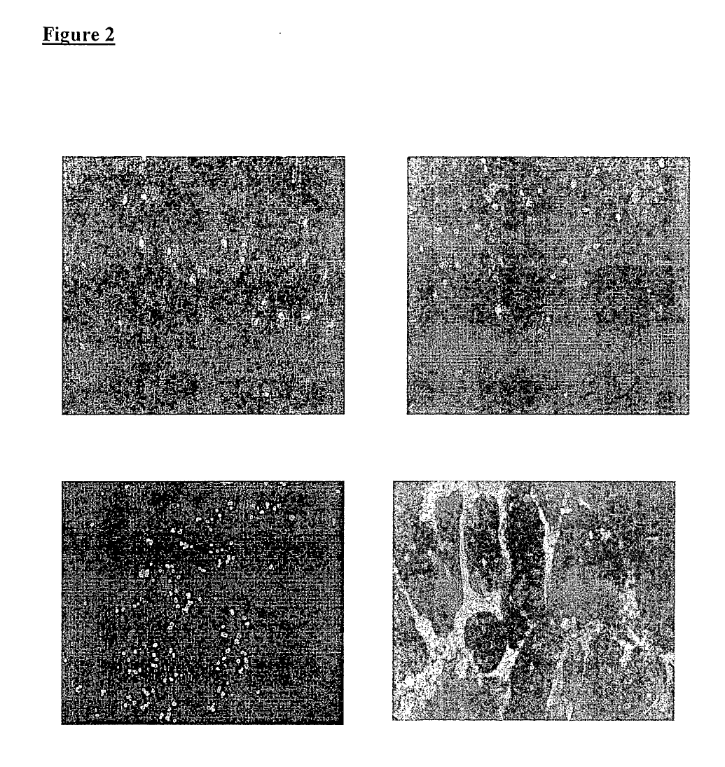

As detailed in Table 2 and FIG. 2, among 10 patients with objective responses 9 are assessable by FISH and 8/9 (88.8%) show increased EGFR gene copy number (median EGFR gene/nucleus ratio 6.80, range 1.65-35); among the 21 non-responder patients, 20 were assessable by FISH and 1/20 (5.0%) had increased EGFR gene copy number (median EGFR gene/nucleus ratio 1.925) and this difference can be found statistically significant.

Among responders, increased EGFR gene copy number can be associated with an EGFR gene/CEP7 ratio >2 in seven out of nine FISH assessable patients, thus indicating an amplification of the EGFR gene employing criteria utilized for HER2 evaluation (Wiley, Diaz, 2004, Jama 291: 2019-2020.). In patients 3 and 9, an EGFR gene/nucleus ratio of 7.10 and 3.38 can be associated with an EGFR gene/CEP7 ratio of 1.46 and 1.19, respectively, thus indicating the presence of extra copies of the entire chromosome 7 (polysomy 7) (FIG. 2C).

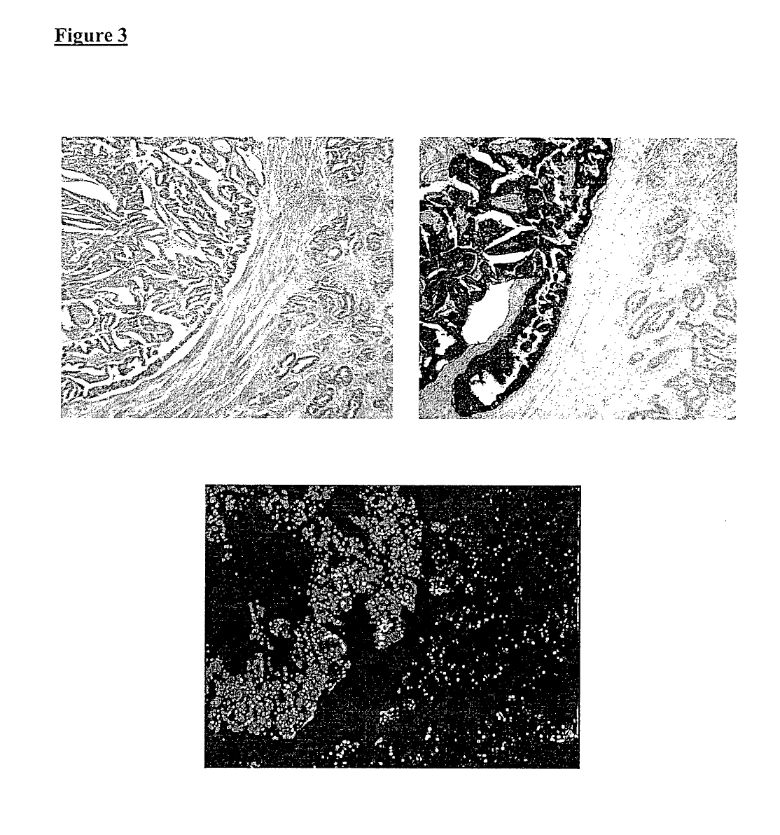

The tumor of patient 10 exhibits a striking amplification of the EGFR gene that can be localized into discrete foci while other malignant areas are frankly disomic. Notably, areas displaying EGFR gene amplification also show intense expression of the EGFR protein assessed by IHC; in contrast, the areas exhibiting disomic EGFR gene do not express the corresponding protein (FIG. 3).

Copy Number Analysis of the EGFR Gene by Quantitative PCR (qPCR)

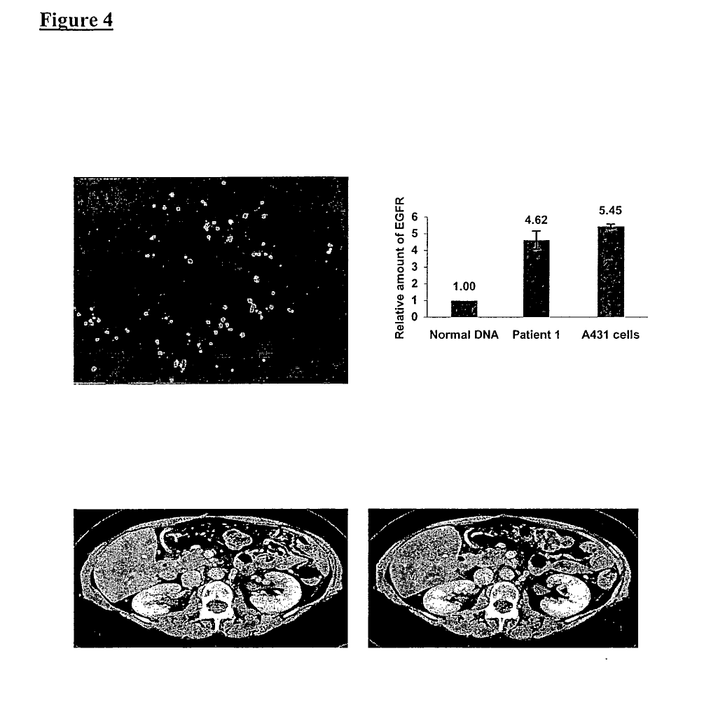

Increased EGFR gene copy number can be observed in patients with response to cetuximab, matuzumab or panitumumab by FISH. To obtain an independent measurement of the status of the EGFR gene locus in tumor specimens, qPCR analysis can be used. An increase in EGFR gene copy number can be observed in patient 1 with responsive disease (FIG. 4). Detection of increased EGFR gene copy number by qPCR in samples from patients with a gene/chromosome ratio below 3 is not conclusive. This is likely due to the limited EGFR gene numbers that cannot be consistently detected with this method as previously reported (Layfield et al., 2003, J Surg Oncol 83: 227-231; Yang et al., 2004, Gut 53(1): 123-129). Additionally, qPCR detection may be negatively affected by the concomitant extraction of normal stromal contaminant DNA that can only be partially avoided during dissection of paraffin embedded samples. On the other hand, in situ analysis of gene copy number such as that obtained by FISH analysis is not affected by these technical limitations. This qPCR gene copy number measurement confirms the amplification

Effects of Cetuximab on Cell Lines with Normal or Increased EGFR Gene Copies

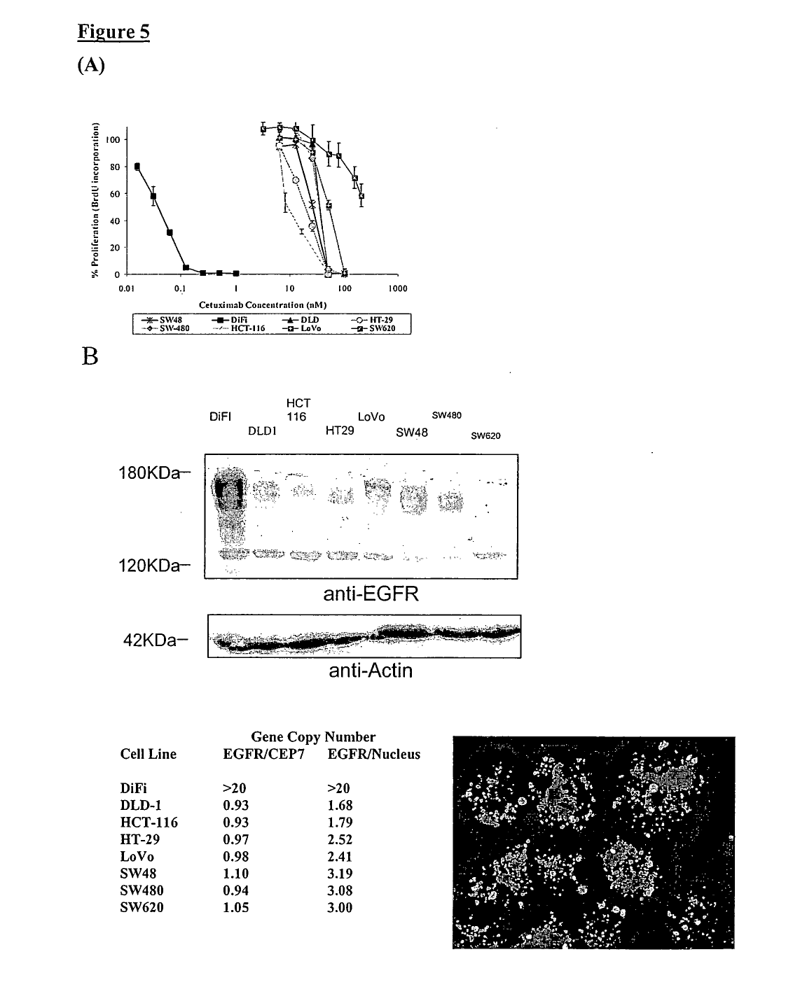

Previous studies using cellular cancer models have suggested that response to cetuximab can be associated with (i) overexpression of the EGFR receptor, (ii) constitutive phosphorylation of the receptor, (iii) amplification of the corresponding gene, and (iv) alteration of other members of the gene family. The present data show that panitumumab, matuzumab or cetuximab responsiveness in mCRC correlates with increased gene copy number of the EGFR locus. This prompted the inventors to assess the effect of cetuximab on a panel of colorectal cancer cell lines displaying normal or increased EGFR gene copy number as measured by FISH (FIG. 5). Cell proliferation measured by the BrdU incorporation assay are evaluated in the presence of increasing cetuximab concentrations. The proliferation of the DiFi cell line that carries the highest copies of the EGFR gene are dramatically inhibited by cetuximab and the concentration of cetuximab that completely impairs proliferation of DiFi cells does not affect cells with not amplified EGFR copy number. Interestingly, the SW620 cell line has 3 copies of the EGFR gene and does not express the EGFR protein as shown by Western blot (FIG. 5). The SW620 cells therefore represent a functional knock out of the EGFR gene and accordingly its proliferation is virtually unaffected by cetuximab.

The term “ErbB receptor antagonist/inhibitor” refers to a biologically effective molecule, which binds and blocks or inhibits the ErbB receptor. Thus, by blocking the receptor the antagonist prevents binding of the ErbB ligand (agonist) and activation of the agonist/ligand receptor complex. ErbB antagonists may be directed to HER1 (ErbB1, EGFR), HER2 (ErbB2) and ErbB3 and ErbB4. Preferred antagonists of the invention are directed to the EGF receptor (EGFR, HER1). The ErbB receptor antagonist may be an antibody an antibody fusion protein (immunoconjugate) or an immunotherapeutically effective fragment of an antibody or an antibody fusion protein. ErbB receptor antagonists, which are preferred according to the present invention, are anti-EGFR antibodies, especially and preferably the anti-EGFR antibodies mentioned above and below: cetuximab, panitumumab and matuzumab in their murine, cimeric or humanized versions including their immunologically effective fragments (Fab, Fv) and immunoconjugates, especially immunocytokines.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. Methods for making monoclonal antibodies include the hybridoma method described by Kohler and Milstein (1975, Nature 256, 495) and in “Monoclonal Antibody Technology, The Production and Characterization of Rodent and Human Hybridomas” (1985, Burdon et al., Eds, Laboratory Techniques in Biochemistry and Molecular Biology, Volume 13, Elsevier Science Publishers, Amsterdam), or may be made by well known recombinant DNA methods (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:58, 1-597 (1991), for example.

The term “chimeric antibody” means antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g.: U.S. Pat. No. 4,816,567; Morrison et al., Proc. Nat. Acad. Sci. USA, 81:6851-6855 (1984)). Methods for making chimeric and humanized antibodies are also known in the art. For example, methods for making chimeric antibodies include those described in patents by Boss (Celltech) and by Cabilly (Genentech) (U.S. Pat. No. 4,816,397; U.S. Pat. No. 4,816,567).

“Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

“Antibody fragments” comprise a portion of an intact antibody, preferably comprising the antigen-binding or variable region thereof. Examples of antibody fragments include Fab, Fab′, F(ab′)2, Fv and Fc fragments, diabodies, linear antibodies, single-chain antibody molecules; and multispecific antibodies formed from antibody fragment(s). An “intact” antibody is one which comprises an antigen-binding variable region as well as a light chain constant domain (CL) and heavy chain constant domains, CH1, CH2 and CH3. Preferably, the intact antibody has one or more effector functions. Papain digestion of antibodies produces two identical antigen-binding fragments, called “Fab” fragments, each comprising a single antigen-binding site and a CL and a CH1 region, and a residual “Fc” fragment, whose name reflects its ability to crystallize readily. The “Fc” region of the antibodies comprises, as a rule, a CH2, CH3 and the hinge region of an IgG1 or IgG2 antibody major class. The hinge region is a group of about 15 amino acid residues which combine the CH1 region with the CH2-CH3 region. The “Fab” fragment also contains the constant domain of the light chain and the first constant domain (CH1) of the heavy chain and has one antigen-binding site only.

“Fab” fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known (see e.g. Hermanson, Bioconjugate Techniques, Academic Press, 1996; U.S. Pat. No. 4,342,566). “Single-chain Fv” or “scFv” antibody fragments comprise the V, and V, domains of antibody, wherein these domains are present in a Single polypeptide chain. Preferably, the Fv polypeptide further comprises a polypeptide linker between the VH and VL domains which enables the scFv to form the desired structure for antigen binding. Single-chain FV antibodies are known, for example, from Plückthun (The Pharmacology of Monoclonal Antibodies, Vol. 113, Rosenburg and Moore eds., Springer-Verlag, New York, pp. 269-315 (1994)), WO93/16185; U.S. Pat. No. 5,571,894; U.S. Pat. No. 5,587,458; Huston et al. (1988, Proc. Natl. Acad. Sci. 85, 5879) or Skerra and Plueckthun (1988, Science 240, 1038).

Although the invention relates preferably to colon or colorectal cancer (CRC) it is principally applicable to other cancers and tumors, which express or overexpress EGFR and occur in patients with different EGFR gene copy numbers and treated with other ErbB antagonists (e.g. lung cancer treated with IRESSA®: e.g. Cancer Biology 2005, 4).

Thereby, the terms “cancer” and “tumor” refer to or describe the physiological condition in mammals that is typically characterized by unregulated cell growth. By means of the pharmaceutical compositions according of the present invention tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. Tumors which can be preferably be treated with the antibody molecules according to the invention are solid tumors or tumor metastases that express ErbB receptors, especially ErbB1 (EGFR) receptors, in high amounts, such as breast cancer, prostate cancer head and neck cancer, SCLC, pancreas cancer.

The term “biologically/functionally effective” or “therapeutically effective (amount)” refers to a drug/molecule which causes a biological function or a change of a biological function in vivo or in vitro, and which is effective in a specific amount to treat a disease or disorder in a mammal, preferably in a human. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer.

The term “immunotherapeutically effective” refers to biological molecules which cause an immune response in a mammal. More specifically, the term refers to molecules which may recognize and bind an antigen. Typically, antibodies, antibody fragments and antibody fusion proteins comprising their antigen binding sites (complementary determining regions, CDRs) are immunotherapeutically effective.

Typically, a therapeutically effective amount of an anti-EGFR antibody or a fragment thereof is an amount such that, when administered in physiologically tolerable composition, is sufficient to achieve a plasma concentration of from about 0.01 microgram (μg) per milliliter (ml) to about 100 μg/ml, preferably from about 1 μg/ml to about 5 μg/ml and usually about 5 μg/ml. Stated differently. the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily for one or several days. A preferred plasma concentration in molarity is from about 2 micromolar (μM) to about 5 millimolar (mM) and preferably, about 100 μM to 1 mM antibody antagonist.

The pharmaceutical compositions of the invention can comprise phrase encompasses treatment of a subject with agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art. The immunotherapeutic agents according to the invention can additionally administered with adjuvants like BCG and immune system stimulators. Furthermore, the compositions may include immunotherapeutic agents or chemotherapeutic agents including such, which contain cytotoxic effective radio-labeled isotopes, or other cytotoxic agents, such as a cytotoxic peptides (e.g. cytokines) or cytotoxic drugs and the like.

Other features and advantages of the present invention will become apparent from the following more detailed Examples, which illustrate, by way of example, the principles of the invention. Especially, specific values or terms indicated above and below, are not limiting the invention and can be extrapolated if a skilled worker sees reason for that.

### EXAMPLES

### Example 1

Patients and Treatment With Anti-EGFR Monoclonal Antibodies

Among patients enrolled at Ospedale Niguarda Ca' Granda into clinical trials of anti-EGFR moAbs panitumumab or cetuximab for treatment of EGFR-expressing mCRC, we evaluated 31 patients with radiologically demonstrated tumor sensitivity or resistance to this therapy (Table 1). Patients were selected based on the availability of sufficient tumour tissue for present studies. All patients had EGFR-expressing mCRC, displaying ≧1% malignant cells stained for EGFR evaluated by IHC using the DAKO EGFRPharmDX kit in central laboratories of each clinical protocol (Cunningham et al., 2004, N Engl J Med 351: 337-345). Cetuximab (chimeric IgG1 moAb; Erbitux®, Merck, Milan, Italy) and panitumumab (fully human IgG2 moAb; Amgen, Thousand Oaks, Calif., USA) both target the ligand-binding domain of the EGFR. Their clinical activities are expected to be comparable except for the reduced incidence of infusion reactions seen with the fully human panitumumab, and thus the patients treated with either moAb are analyzed together in this study. Treatment with anti-EGFR moAbs consisted of cetuximab monotherapy (n=12), cetuximab plus irinotecan (Camptò®; Aventis, Milan, Italy) based chemotherapy (n=9), or panitumumab monotherapy (n=10). In particular, single agent cetuximab (400 mg/m² iv loading dose and then 250 mg/m² weekly until progression) was given either as first-line therapy in EMR 202-600 phase-II trial or as third-line in the monotherapy arm of BOND phase II trial for irinotecan-refractory patients. Cetuximab (same dose and schedule as in monotherapy) plus irinotecan (same doses and schedules to which mCRC were individually demonstrated to be resistant) were given until progression as third-line therapy in the combination arm of BOND trial and in MABEL phase-II trial for irinotecan-refractory patients. In the latter protocols, refractoriness to irinotecan was defined as documented disease progression during or within 3 months after irinotecan regimen. Single agent panitumumab (6 mg/kg iv every 2 weeks until progression) was given as third-line or fourth-line therapy for patients resistant to both oxaliplatin- and irinotecan-containing regimens in the phase III ABX-EGF 20020408 and cross-over ABX-EGF 20020194 trials. The Institutional Ethics Committee approved the treatment protocols, and patients gave written informed consent for analysis of EGFR as well as for receiving study therapy. Tumor response was evaluated with consistent imaging techniques (CT or MRI) employing RECIST (Response Evaluation Criteria in Solid Tumors) criteria by institutional as well as independent radiologists according to clinical protocols.

### Example 2

Mutational Analysis

DNA was extracted from paraffin embedded samples. For each patient, 10 [symbol] sections were prepared. An additional representative section was deparaffinized, stained with hematoxylin-eosin and analyzed for detailed morphology. Regions displaying tumor tissues were marked and the tissue was extracted with 0.2M NaOH/1 mM EDTA and then neutralized with 100 mM Tris-TE. After extraction DNA was purified using Qiagen PCR Purification Kit (Cat. No. 28104) following manufacturer instructions. Exon specific and sequencing primers were designed using Primer3 software (http://frodo.wi.mit.edu/cgi-bin/primer3/primer3_www.cgi) and synthesized by Invitrogen™. Primer sequences were: Forward, reverse and sequencing primers for each exon were as follows:

EGFR-Ex18
GCTGAGGTGACCCTTGTCTC; ACAGCTTGCAAGGACTCTGG;
TGGAGCCTCTTACACCCAGT;
EGFR-Ex19
CCCAGTGTCCCTCACCTTC; CCACACAGCAAAGCAGAAAC;
GCTGGTAACATCCACCCAGA;
EGFR-Ex21
TGATCTGTCCCTCACAGCAG; TCAGGAAAATGCTGGCTGAC;
TTCAGGGCATGAACTACTTGG;
P13K CA-Ex9
GGGAAAAATATGACAAAGAAAGC; CTGAGATCAGCCAAATTCAGTT;
TAGCTAGAGACAATGAATTAAGGGAAA;
P13K CA-Ex20
CTCAATGATGCTTGGCTCTG; TGGAATCCAGAGTGAGCTTTC;
TTGATGACATTGCATACATTCG
Ras ex2
GGTGGAGTATTTGATAGTGTATTAACC; AGAATGGTCCTGCACCAGTA
A; TCATTATTTTTATTATAAGGCCTGCTG.

Conditions to amplify exon-specific regions by PCR from tumor genomic DNA and to identify mutations have been previously described (Bardelli et al., 2003, Science 300: 949). PCR was carried out in a volume of 20 μL using a touchdown PCR program as previously described (Pao et al., 2004, Proc Natl Acad Sci USA 101: 13306-13311). Purified PCR products were sequenced using BigDye® Terminator v3.1 Cycle Sequencing Kit (Applied Biosystems) and analyzed with a 3730 ABI capillary electrophoresis system. Mutational analysis was carried out as previously described. Tumor tissue from patient 13 was limited in quantity and mutational analysis was not technically possible for all exons.

### Example 3

Analysis of EGFR Gene by Fluorescent In Situ Hybridization (FISH)

Tissue sections were treated following the procedure used for Her2 FISH detection Kit (Dakocytomation, Glostrup, DK). Samples were placed in a pretreatment solution for 30 min at 96° C. and then digested with pepsin solution for 30 min at room temperature. Dual-color, dual-target FISH assays were performed using the LSI EGFR Spectrum Orange/CEP7 Spectrum Green Probe (Vysis, Downers Grove, Ill.). Briefly tissue sections, covered with 10 μL probe solution, were incubated at 75° C. for 5 min to co-denature the EGFR and CEP 7 probes and allowed to hybridize overnight at 37° C. Both co-denaturation and hybridization were performed sequentially in a microprocessor-controlled system (Hybridizer, Dakocytomation, Glostrup, DK). Post-hybridization stringency wash was performed in water bath at 65° C. for 10 min. After washing twice and drying at room temperature for 15 min, tissue sections were covered with 4′6-diamidino-2-phenylindole (DAPI II, Vysis) for chromatin counterstaining and examined by microscopy. Analysis was performed with a fluorescence microscope (Zeiss Axioskop, Gottingen, Germany) equipped with the Chromowin workstation (Amplimedical, Milan, Italy). The EGFR gene was visualized as a red signal with a tetramethyl-rhodamine isothiocyanate (TRITC) filter, the chromosome 7 α-centromeric (CEP7) sequence as green signal with a fluorescein isothiocyanate (FITC) filter and the nuclei as a blue signal with a DAPI filter. Representative images of each specimen were acquired with a Hamamatsu C5895 chilled CCD camera (Upstate Technical Equipment Co., New York, USA) in monochromatic layers that were subsequently merged by the Casti Imaging FISH Multicolor software (Amplimedical). Two independent observers (SMV and RB) scored at least 200 non-overlapping interphase nuclei using predefined scoring guidelines. The observers were blinded to clinical characteristics of the patients and each other's assessment and scoring of the specimens. In each nucleus, the number of copies of EGFR and chromosome 7 probes was assessed independently. The EGFR gene status was scored as EGFR/nucleus and EGFR/CEP7 ratios. Normal controls consisted of cultured retinal pigment epithelial (RPE) cell line and normal colorectal mucosa contiguous to individual malignancies. Amplified EGFR gene control consisted of A431 human epidermoid carcinoma cell line. Increased EGFR gene copy number was arbitrarily defined as EGFR gene copy number/nucleus ≧3. Specimens from patients 4 and 15 were available only as 10μ sections and despite multiple attempts, FISH analysis was not conclusive due to excessive tissue thickness.

### Example 4

Analysis of EGFR Gene by Quantitative Polymerase Chain Reaction (qPCR)

The number of copies corresponding to the EGFR locus was determined by real time PCR using an ABI PRISM® 7900HT apparatus (Applied Biosystems). DNA content was normalized to that of Line-1, a repetitive element for which copy numbers per diploid genome are similar among all human cells (normal or malignant) as previously described (Wang et al., 2002, Proc Natl Acad Sci USA 99: 16156-16161). Copy number changes were calculated by using the formula 2^{(Dt-Dline)-(Nt-Nline)} where Dt is the average threshold cycle number observed for the experimental primer in DNA extracted from tumor cells, and an experimental primer Dline is the average threshold cycle number observed for the Line-1 primer in DNA extracted from tumor cell and Nt is the threshold cycle number observed for in the normal reference DNA extracted from RPE cells, Nline is the threshold cycle number observed for a Line-1 primer in the normal reference DNA extracted from RPE cells. Conditions for amplification were as follows: one cycle of 95° C. for 10 min, followed by 45 cycles of 95° C. for 15 sec, 60° C. for 1 min. Threshold cycle numbers were obtained by using the ABI PRISM® 7900HT Sequence Detection System software. PCRs for each primer set were performed in triplicate and threshold cycle numbers were averaged. Primers (designed to span a 100 to 200-bp non-repetitive region) for the EGFR gene were: Forward GAATTCGGATGCAGAGCTTC and Reverse GACATGCTGCGGTGTTTTC. Primers for the Line-1 repetitive element were: Forward AAAGCCGCTCAACTACATGG and Reverse TGCTTTGAATGCGTCCCAGAG.

### Example 5

Cell Proliferation Inhibition Assay and Western Blotting

Colorectal cancer cell lines (HT-29, HCT-116, DLD-1, SW48, SW480, and LoVo cells) were from ATCC repository; DiFi cells were a gift of Jose Baselga, Vail d'Hebron University, Barcelona, E). Cells were grown in DMEM supplemented with 10% fetal calf serum (FCS) and antibiotics, except for DiFi cells which were grown in F-12 Medium supplemented with 10% FCS and antibiotics. For cell proliferation inhibition assay, cells were grown in DMEM supplemented with 2% FBS in 96-well black plates (Culture Plate™ 96F Packard Bioscience) and incubated for 5 days with 0.01-100 nM cetuximab (purchased from Komtur Pharmaceuticals, Freiburg, D). Cell proliferation was measured by incorporation of BrdU using a chemiluminescent ELISA method (Roche Cat. No. 1 669 915).

The cell seeding density per well was as follows: DiFi, 4000; LoVo, 4000; DLD, 500; HCT116, 1000; HT29, 1000; SW480, 1000; SW387, 4000; SW48, 500; SW620, 500. The BrdU assay was carried out according to the manufacturer's instructions and terminated 20 hrs after addition of the labeling solution. Three separate experiments in triplicate were set up for each cell line. The percentage of cell proliferation at each cetuximab concentration (Test) was calculated using the following formula: (Test−blank)/(Control−blank)×100, where control indicates cells grown in medium only (no drug) and blank indicates cells grown in 0.02% Triton X in DMEM. Western blotting was carried out as previously described (Lynch and Yang, 2002, Semin Oncol 29: 47-50).

TABLE 1
Relevant clinical characteristics and EGFR gene molecular
alterations in tumors of patients with mCRC

| | | Tumor response | | | |
|---|---|---|---|---|---|
| | | | Duration of | Molecular analysis of EGFR | |
| Patient number | Therapy with | Best | response | COPY | |
| and UPN | anti-EGFR antibody | response | (weeks) | NUMBER^{b} | SEQUENCE* |
| 1 - MR120653 | Cetuximab and CT^{a} | PR | 48 | Increased | WT |
| 2 - LM090846 | Cetuximab and CT | PR | 36 | Increased | WT |
| 3 - RP180336 | Cetuximab and CT | PR | 36+ | Increased | WT |
| 4 - LS250848 | Cetuximab | PR | 30 | Not evaluable^{c} | WT |
| 5 - AC201146 | Panitumumab | PR | 33 | Increased | WT |
| 6 - GL240243 | Panitumumab | PR | 24 | Increased | WT |
| 7 - FC151048 | Panitumumab | PR | 16 | Increased | WT |
| 8 - PA260526 | Cetuximab | PR | 16+ | Normal | WT |
| 9 - AM180627 | Panitumumab | PR | 12+ | Increased | WT |
| 10 - GM281120 | Cetuximab | PR | 8+ | Increased | WT |
| 11 - SM070445 | Cetuximab | SD | 30 | Normal | WT |
| 12 - LC280946 | Cetuximab and CT | SD | 24 | Normal | WT |
| 13 - AG080530 | Cetuximab and CT | SD | 24 | Normal | Exon 21G857R |
| 14 - MM180625 | Cetuximab | SD | 36+ | Normal | WT |
| 15 - GM160553 | Panitumumab | SD | 32 | Not evaluable^{c} | WT |
| 16 - CC090234 | Panitumumab | SD | 16+ | Normal | WT |
| 17 - GT030547 | Cetuximab | PD | N.A. | Increased | WT |
| 18 - SM140851 | Cetuximab and CT | PD | N.A. | Normal | WT |
| 19 - AC230643 | Cetuximab | PD | N.A. | Normal | WT |
| 20 - DS010731 | Cetuximab and CT | PD | N.A. | Normal | WT |
| 21 - RV110964 | Cetuximab and CT | PD | N.A. | Normal | WT |
| 22 - CC041133 | Cetuximab | PD | N.A. | Normal | WT |
| 23 - GT050933 | Cetuximab | PD | N.A. | Normal | WT |
| 24 - RT161027 | Cetuximab | PD | N.A. | Normal | WT |
| 25 - CB280630 | Cetuximab | PD | N.A. | Normal | WT |
| 26 - FL020230 | Cetuximab | PD | N.A. | Normal | WT |
| 27 - PC020849 | Panitumumab | PD | N.A. | Normal | WT |
| 28 - CF141238 | Panitumumab | PD | N.A. | Normal | WT |
| 29 - WB030428 | Cetuximab | PD | N.A. | Normal | WT |
| 30 - GA240151 | Panitumumab | PD | N.A. | Normal | WT |
| 31 - IM100640 | Panitumumab | PD | N.A. | normal | WT |

^{a}Chemotherapy (CT) consisted of irinotecan-based treatment (see text for details);
^{b}increased EGFR gene copy number consisted of balanced polysomy in case 3 and 9 and
gene amplification in the others (see results);
^{c}multiple FISH attempts were inconclusive for technical reasons (see Methods). FISH
fluorescent In situ-hybridization; PR, partial response; SD, stable disease; PD,
progressive disease; UPN, unique patient number; WT, wild type; + denotes maintained
response at the time of submitting this article (February 2005).
*Mutational status of the EGFR gene, exons 18, 19 and 21.

Additional clinical characteristics of patients with mCRC evaluated in this study

| Patient number | | | | | |
|---|---|---|---|---|---|
| and UPN | Sex | Age | PS^{a} | No | Regimens for metastatic disease^{b} |
| 1 - MR120653 | F | 52 | 0 | 3 | 5-FU/FA, FOLFOX, Irinotecan |
| 2 - LM090846 | M | 59 | 0 | 3 | 5-FU/FA, FOLFOX, FOLFIRI |
| 3 - RP180336 | M | 69 | 0 | 2 | FOLFOX, FOLFIRI |
| 4 - LS250848 | M | 57 | 1 | 3 | 5-FU/FA, FOLFOX, FOLFIRI |
| 5 - AC201146 | M | 59 | 0 | 3 | FOLFOX, Capecitabine, FOLFIRI |
| 6 - GL240243 | F | 62 | 1 | 2 | FOLFOX, FOLFIRI |
| 7 - FC151048 | M | 57 | 1 | 2 | FOLFIRI, FOLFOX |
| 8 - PA260526 | M | 79 | 1 | 0 | Not applicable |
| 9 - AM180627 | F | 78 | 1 | 3 | FOLFOX, Capecitabine, FOLFIRI |
| 10 - GM281120 | M | 85 | 1 | 0 | Not applicable |
| 11 - SM070445 | M | 60 | 0 | 1 | Irinotecan |
| 12 - LC280946 | M | 59 | 0 | 2 | FOLFOX, FOLFIRI |
| 13 - AG080530 | M | 75 | 0 | 2 | FOLFOX, FOLFIRI |
| 14 - MM180625 | F | 80 | 1 | 0 | Not applicable |
| 15 - GM160553 | F | 52 | 0 | 4 | FOLFOX, Irinotecan, Capecitabine, FOLFIRI |
| 16 - CC090234 | M | 71 | 1 | 2 | FOLFOX, FOLFIRI |
| 17 - GT030547 | M | 58 | 0 | 0 | Not applicable |
| 18 - SM140851 | M | 54 | 1 | 2 | FOLFOX, Irinotecan + weekly high-dose 5-FU/AF |
| 19 - AC230643 | M | 62 | 1 | 0 | Not applicable |
| 20 - DS010731 | M | 74 | 0 | 3 | Irinotecan + Capecitabine, FOLFOX, FOLFIRI |
| 21 - RV110964 | M | 41 | 0 | 3 | FOLFOX, FOLFIRI, Irinotecan |
| 22 - CC041133 | F | 72 | 1 | 0 | Not applicable |
| 23 - GT050933 | M | 72 | 1 | 0 | Not applicable |
| 24 - RT161027 | M | 78 | 1 | 0 | Not applicable |
| 25 - CB280630 | F | 75 | 1 | 0 | Not applicable |
| 26 - FL020230 | M | 75 | 1 | 0 | Not applicable |
| 27 - PC020849 | M | 56 | 1 | 1 | Oxaliplatin-Irinotecan-5-FU/FA |
| 28 - CF141238 | F | 67 | 0 | 2 | FOLFOX, FOLFIRI |
| 29 - WB030428 | M | 77 | 1 | 0 | Not applicable |
| 30 - GA240151 | M | 54 | 0 | 2 | FOLFOX, FOLFIRI |
| 31 - IM100640 | F | 65 | 1 | 3 | 5-FU/FA, FOLFOX, FOLFIRI |

No: Number of prior chemotherapy regimens for metastatic disease
^{a}Performance Status (ECOG) at the time of starting anti-EGFR monoclonal antibody therapy.
^{b}Chemotherapy regimens consisted of: 5-FU/FA: 5-fluorouracil + folinic acid (various
schedules); FOLFOX: Oxaliplatin + 5-fluorouracil + folinic acid; FOLFIRI: Irinotecan +
5-fluorouracil + folinic acid.

TABLE 2
Molecular alterations found in tumors of patients with mCRC

| | | Gene copy number | | Mutational analysis | | | | | | |
|---|---|---|---|---|---|---|---|---|---|---|
| | Tumor | EGFR | EGFR | EGFR | EGFR | EGFR | Ras | PI₃K | PI₃K | BRAF |
| Patient UPN | response | gene/CEP7 | gene/nucleus | Exon-18 | Exon-19 | Exon-21 | Exon-2 | Exon-9 | Exon-20 | Exon-15 |
| 1 - MR120653 | PR | 3.37 | 7.90 | WT | WT | WT | WT | E545K | WT | WT |
| 2 - LM090846 | PR | 2.28 | 5.70°° | WT | WT | WT | WT | WT | WT | WT |
| 3 - RP180336 | PR | 1.42 | 7.10 | WT | WT | WT | WT | WT | WT | WT |
| 4 - LS250848 | PR | n.e.^{a} | n.e.^{a} | WT | WT | WT | WT | WT | WT | WT |
| 5 - AC201146 | PR | 2.50 | 4.80 | WT | WT | WT | WT | WT | WT | WT |
| 6 - GL240243 | PR | 2.13 | 6.80 | WT | WT | WT | G13D | WT | WT | WT |
| 7 - FC151048 | PR | 3.27 | 8.20 | WT | WT | WT | G12D | WT | WT | WT |
| 8 - PA260526 | PR | 1.03 | 1.65 | WT | WT | WT | WT | WT | WT | WT |
| 9 - AM180627 | PR | 1.19 | 3.38 | WT | WT | WT | WT | WT | WT | WT |
| 10 - GM281120 | PR | 8.75 focal | 35 focal | WT | WT | WT | WT | WT | WT | WT |
| | | clustered | clustered | | | | | | | |
| 11 - SM70445 | SD | 0.98 | 1.8 | WT | WT | WT | WT | WT | WT | WT |
| 12 - LC280946 | SD | 1.05 | 1.9 | WT | WT | WT | WT | WT | WT | WT |
| 13 - AG080530 | SD | 0.95 | 1.75 | WT | WT | G857R | WT | WT | WT | n.e.^{a} |
| 14 - MM180625 | SD | 1.06 | 1.80 | WT | WT | WT | WT | WT | WT | WT |
| 15 - GM160553 | SD | n.e.^{a} | n.e.^{a} | WT | WT | WT | G13D | WT | WT | WT |
| 16 - CC090234 | SD | 1.04 | 1.88 | WT | WT | WT | G12V | WT | WT | WT |
| 17 - GT030547 | PD | 4.68 | 20.2 | WT | WT | WT | WT | WT | H1047R | WT |
| | | clustered | clustered | | | | | | | |
| 18 - SM140851 | PD | 1.04 | 2.00 | WT | WT | WT | G13D | WT | WT | WT |
| 19 - AC230643 | PD | 0.70 | 1.72 | WT | WT | WT | WT | WT | WT | WT |
| 20 - DS010731 | PD | 0.99 | 1.95 | WT | WT | WT | G12V | WT | WT | WT |
| 21 - RV110964 | PD | 0.95 | 2.00 | WT | WT | WT | WT | WT | WT | WT |
| 22 - CC041133 | PD | 1.00 | 1.90 | WT | WT | WT | G12S | WT | WT | WT |
| 23 - GT050933 | PD | 1.20 | 2.10 | WT | WT | WT | WT | WT | WT | WT |
| 24 - RT161027 | PD | 1.16 | 1.98 | WT | WT | WT | G12D | WT | WT | WT |
| 25 - CB280630 | PD | 0.90 | 1.75 | WT | WT | WT | WT | WT | WT | WT |
| 26 - FL020230 | PD | 0.96 | 1.85 | WT | WT | WT | G12D | WT | WT | WT |
| 27 - PC020849 | PD | 0.91 | 1.70 | WT | WT | WT | WT | WT | WT | WT |
| 28 - CF141238 | PD | 1.02 | 2.00 | WT | WT | WT | G13D | WT | WT | WT |
| 29 - WB030428 | PD | 1.00 | 2.05 | WT | WT | WT | WT | WT | WT | WT |
| 30 - GA240151 | PD | 1.03 | 2.00 | WT | WT | WT | WT | WT | WT | WT |
| 31 - IM100640 | PD | 1.18 | 2.10 | WT | WT | WT | WT | WT | H1047R | E599V |

^{a}FISH and mutational analysis measurements were inconclusive for technical reasons (see Methods); WT, Wilde Type; PR, partial response; SD, stable disease; PD, progressive disease; UPN, unique patient number; n.e., not evaluable.
°°Increased EGFR gene copy number was demonstrated both in primary colorectal tumor prior to moAb treatment and in liver metastasis obtained at the time of progressive disease after moAb treatment.

1. An in vitro method for detecting and analyzing whether a patient suffering from a cancer, which overexpresses EGF receptor (EGFR), responds positively to the administration of an anti-EGFR antibody or an immunologically effective fragment thereof, the method comprising determining in vitro the EGFR gene copy number in a probe of tumor cells obtained from said patient and selecting said patient for administration with said anti-EFFR antibody if the tumor cells of said patient display an amplified copy number of the EGFR gene.

2. A method of claim 1, wherein the EGFR gene copy number is measured as ratio of the number of EGFR genes per nucleus.

3. A method of claim 2, wherein said ratio is in the range between 4.0 and 8.2.

4. A method of claim 2, wherein said ratio is in the range between 5.7 and 7.1.

5. A method of claim 1, wherein the EGFR gene copy number is measured as ratio of the number of EGFR genes per CEP7.

6. A method of claim 5, wherein said ratio is >2.

7. A method according to claim 1, wherein the EGFR gene copy number is measured by FISH analysis (fluorescence in situ hybridization).

8. A method according to claim 1, wherein said amplified EGFR gene copy number is specific for said tumor.

9. A method according to claim 1, wherein the amplified EGFR gene copy number is specific for the individual cancer tissue profile of the patient.

10. A method of claim 9, wherein said individual cancer tissue profile underlies furthermore molecular alteration.

11. A method of claim 10, wherein said molecular alteration is a point mutation within the EGFR gene.

12. A method according to claim 1, wherein said anti-EGFR antibody is selected from the group consisting of cetuximab (mAb c225), matuzumab (mAb h425) and panitumumab (mAb ABX) or their particular murine, chimeric or humanized versions.

13. A method according to claim 1, wherein the cancer is colorectal cancer (CRC), lung cancer, head and neck cancer and breast cancer.

14. Use of an anti-EGFR antibody, or an immunologically effective fragment thereof, for the manufacture of a medicament for the treatment of cancer in a patient, wherein said cancer overexpresses EGFR and displays an amplified EGFR gene copy number.

15. Use of claim 14, wherein said EGFR gene copy number is measured as ratio of the number of EGFR genes per nucleus, and the value of this ratio is in the range between 4.0 and 8.2.

16. Use of claim 15, wherein the value of said ratio is in the range between 5.7 and 7.1.

17. Use according to claim 14, wherein the treatment of said cancer is more effective compared to the treatment of a cancer patient with the same antibody in the same dose, wherein the cancer cells do not display an amplified EGFR copy number.

18. Use according to claim 14, wherein said amplified EGFR gene copy number is specific for said tumor.

19. Use according to claim 14, wherein the amplified EGFR gene copy number is specific for the individual cancer tissue profile of the patient.

20. Use of claim 19, wherein said individual cancer tissue profile underlies genetic mutations.

21. Use of claim 14, wherein said EGFR expressing tumor is colorectal cancer (CRC), lung cancer, breast cancer or head and neck cancer.

22. Use according to claim 14, wherein said anti-EGFR antibody is selected from the group consisting of cetuximab (mAb c225), matuzumab (mAb h425) and panitumumab (mAb ABX), or their particular murine, chimeric or humanized versions.

23. Method for detecting and measuring in vitro the EGFR gene copy number of tumor tissue, which overexpresses EGFR, by using fluorescent in situ hybridization (FISH) in an assay for determining the response of a cancer patient to the administration with an anti-EGFR antibody.

24. A method of treating cancer which overexpresses EGFR and displays an amplified EGFR gene copy number comprising administering an anti-EGFR antibody, or an immunologically effective fragment thereof.
